(19) United States
(12) Patent Application Publication
(10) Pub. No.: US 2019/0001090 A1
(43) Pub. Date: Jan. 3, 2019

(54) PRESSURE SUPPORT DEVICE INCLUDING SENSOR TO DETECT NON-SLEEP DISORDERED BREATHING CONDITIONS

(71) Applicant: KONINKLIJKE PHILIPS N.V., Eindhoven (NL)

(72) Inventor: Eric Alan HIGGINS, Eindhoven (NL)

(73) Assignee: Koninklijke Philips N.V., Eindhoven (NL)

(21) Appl. No.: 16/065,144

(22) PCT Filed: Dec. 12, 2016
(86) PCT No.: PCT/IB2016/057527

§ 371 Date: Jun. 22, 2018

Related U.S. Application Data
(60) Provisional application No. 62/270,755, filed on Dec. 22, 2015.

(51) Int. Cl. A61M 16/00; A61B 5/1455; A61B 5/00; A61B 5/145; A61M 16/20; G16H 50/30

(52) U.S. Cl. CPC A61M 16/0051; A61M 2205/583; A61B 5/7275; A61B 5/746; A61B 5/14546; A61B 5/7405; A61B 5/742; A61M 16/0069; A61M 16/026; A61M 16/202; G16H 50/30; A61M 2016/0039; A61M 2205/3303; A61M 2205/3569; A61M 2205/3592; A61M 2205/52; A61M 2205/6018; A61M 2205/6063; A61M 2205/581; A61B 5/14552

(57) ABSTRACT

A pressure support device (4) includes a sensor (32;32′) structured to gather data on one or more medical parameters associated with a non-sleep disordered breathing medical condition, a processing unit (31;31′) structured to analyze a risk of the non-sleep disordered breathing medical condition, and an indicator (38) structured to alert the patient when the risk exceeds a predetermined threshold level.

### CROSS-REFERENCE TO RELATED APPLICATIONS

This patent application claims the priority benefit under 35 U.S.C. § 119(e) of U.S. Provisional Application No. 62/270,755 filed on Dec. 22, 2015, the contents of which are herein incorporated by reference.

### BACKGROUND OF THE INVENTION

### 1. Field of the Invention

The present invention pertains to a pressure support device, and, in particular, to a pressure support device including a sensor to detect non-sleep disordered breathing conditions.

### 2. Description of the Related Art

Many individuals suffer from disordered breathing during sleep. Sleep apnea is a common example of such sleep disordered breathing suffered by millions of people throughout the world. One type of sleep apnea is obstructive sleep apnea (OSA), which is a condition in which sleep is repeatedly interrupted by an inability to breathe due to an obstruction of the airway; typically the upper airway or pharyngeal area. Obstruction of the airway is generally believed to be due, at least in part, to a general relaxation of the muscles which stabilize the upper airway segment, thereby allowing the tissues to collapse the airway. Another type of sleep apnea syndrome is a central apnea, which is a cessation of respiration due to the absence of respiratory signals from the brain's respiratory center. An apnea condition, whether OSA, central, or mixed, which is a combination of OSA and central, is defined as the complete or near cessation of breathing, for example a 90% or greater reduction in peak respiratory air-flow.

Those afflicted with sleep apnea experience sleep fragmentation and complete or nearly complete cessation of ventilation intermittently during sleep with potentially severe degrees of oxyhemoglobin desaturation. These symptoms may be translated clinically into extreme daytime sleepiness, cardiac arrhythmias, pulmonary-artery hypertension, congestive heart failure and/or cognitive dysfunction. Other consequences of sleep apnea include right ventricular dysfunction, carbon dioxide retention during wakefulness, as well as during sleep, and continuous reduced arterial oxygen tension. Sleep apnea sufferers may be at risk for excessive mortality from these factors as well as by an elevated risk for accidents while driving and/or operating potentially dangerous equipment.

Even if a patient does not suffer from a complete or nearly complete obstruction of the airway, it is also known that adverse effects, such as arousals from sleep, can occur where there is only a partial obstruction of the airway. Partial obstruction of the airway typically results in shallow breathing referred to as a hypopnea. A hypopnea is typically defined as a 50% or greater reduction in the peak respiratory airflow. Other types of sleep disordered breathing include, without limitation, upper airway resistance syndrome (UARS) and vibration of the airway, such as vibration of the pharyngeal wall, commonly referred to as snoring. Thus, in diagnosing a patient with a breathing disorder, such as OSA, central apneas, or UARS, it is important to detect accurately the occurrence of apneas and hypopneas of the patient.

It is well known to treat sleep disordered breathing by applying a positive airway pressure (PAP) to the patient's airway using an airway pressure support system that typically includes a mask, a pressure generating device, and a conduit to deliver positive pressure breathing gas from the pressure generating device to the patient through the mask. This positive pressure effectively “splints” the airway, thereby maintaining an open passage to the lungs. In one type of PAP therapy, known as continuous positive airway pressure (CPAP), the pressure of gas delivered to the patient is constant throughout the patient's breathing cycle. It is also known to provide a positive pressure therapy in which the pressure of gas delivered to the patient varies with the patient's breathing cycle, or varies with the patient's effort, to increase the comfort to the patient. This pressure support technique is referred to as bi-level pressure support, in which the inspiratory positive airway pressure (IPAP) delivered to the patient is higher than the expiratory positive airway pressure (EPAP).

Pressure support therapies often involve a patient's regular interaction with a pressure support device, typically at night to turn on the device and in the morning to turn off the device. While pressure support therapies and pressure support therapy devices are effective in treating sleep disordered breathing conditions, there remains significant potential to expand the functionality of pressure support devices.

### SUMMARY OF THE INVENTION

In one embodiment, a pressure support device includes: a sensor structured to gather data on one or more medical parameters associated with a non-sleep disordered breathing medical condition; a processing unit structured to analyze a risk to the patient of the non-sleep disordered breathing medical condition; and an indicator structured to alert the patient when the risk exceeds a predetermined threshold level.

### BRIEF DESCRIPTION OF THE DRAWINGS

FIG. 1 is a schematic diagram of a pressure support system adapted to provide a regimen of respiratory therapy to a patient according to an exemplary embodiment of the disclosed concept;

FIG. 2 is a schematic diagram of a scanning unit according to an exemplary embodiment of the disclosed concept;

FIG. 3 is a flowchart of a method of determining risk according to an exemplary embodiment of the disclosed concept;

FIG. 4 is a view of a pressure support device in accordance with an exemplary embodiment of the disclosed concept;

FIG. 5 is a schematic diagram of a pressure support system adapted to provide a regimen of respiratory therapy to a patient according to another exemplary embodiment of the disclosed concept;

FIG. 6 is a scanning unit according to another exemplary embodiment of the disclosed concept;

FIG. 7 is a view of a pressure support device according to another exemplary embodiment of the disclosed concept; and

FIG. 8 is a flowchart of a method of retrieving information from a label according to an exemplary embodiment of the disclosed concept.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

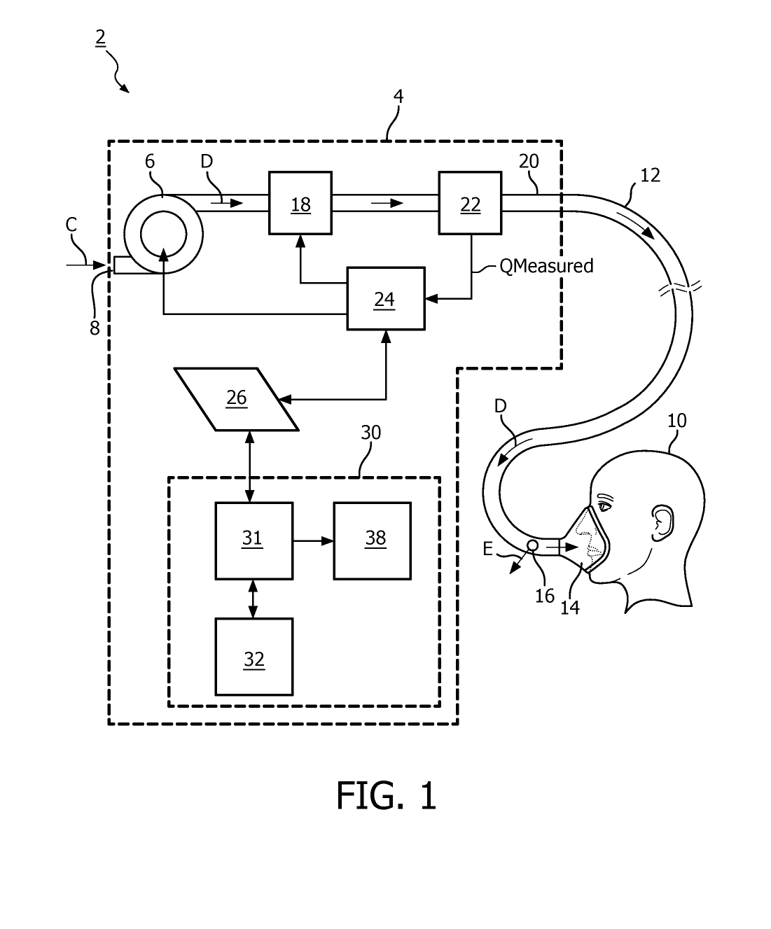

FIG. 1 is a schematic diagram of an airway pressure support system 2 according to one particular, non-limiting exemplary embodiment in which the present invention may be implemented. Referring to FIG. 1, airway pressure support system 2 includes a pressure support device 4 which houses a gas flow generator 6, such as a blower used in a conventional CPAP or bi-level pressure support device. Gas flow generator 6 receives breathing gas, generally indicated by arrow C, from the ambient atmosphere through a filtered air inlet 8 (described in greater detail herein) provided as part of pressure support device 4, and generates a flow of breathing gas therefrom for delivery to an airway of a patient 10 at relatively higher and lower pressures, i.e., generally equal to or above ambient atmospheric pressure. In the exemplary embodiment, gas flow generator 6 is capable of providing a flow of breathing gas ranging in pressure from 3-30 cmH2O. The pressurized flow of breathing gas from gas flow generator 6, generally indicated by arrow D, is delivered via a delivery conduit 12 to a breathing mask or patient interface 14 of any known construction, which is typically worn by or otherwise attached to patient 10 to communicate the flow of breathing gas to the airway of patient 10. Delivery conduit 12 and patient interface device 14 are typically collectively referred to as a patient circuit.

Pressure support system 2 shown in FIG. 1 is what is known as a single-limb system, meaning that the patient circuit includes only delivery conduit 12 connecting patient 10 to pressure support system 2. As such, an exhaust vent 16 is provided in delivery conduit 12 for venting exhaled gases from the system as indicated by arrow E. It should be noted that exhaust vent 16 can be provided at other locations in addition to or instead of in delivery conduit 12, such as in patient interface device 14. It should also be understood that exhaust vent 16 can have a wide variety of configurations depending on the desired manner in which gas is to be vented from pressure support system 2.

The present invention also contemplates that pressure support system 2 can be a two-limb system, having a delivery conduit and an exhaust conduit connected to patient 10. In a two-limb system (also referred to as a dual-limb system), the exhaust conduit carries exhaust gas from patient 10 and includes an exhaust valve at the end distal from patient 10. The exhaust valve in such an embodiment is typically actively controlled to maintain a desired level or pressure in the system, which is commonly known as positive end expiratory pressure (PEEP).

Furthermore, in the illustrated exemplary embodiment shown in FIG. 1, patient interface 14 is a nasal/oral mask. It is to be understood, however, that patient interface 14 can include a nasal mask, nasal pillows, a tracheal tube, an endotracheal tube, or any other device that provides a suitable gas flow communicating function. Also, for purposes of the present invention, the phrase “patient interface” can include delivery conduit 12 and any other structures that couple the source of pressurized breathing gas to patient 10.

In the illustrated embodiment, pressure support system 2 includes a pressure controller in the form of a valve 18 provided in internal delivery conduit 20 provided in a housing of pressure support device 4. Valve 18 controls the pressure of the flow of breathing gas from gas flow generator 6 that is delivered to patient 10. For present purposes, gas flow generator 6 and valve 18 are collectively referred to as a pressure generating system because they act in concert to control the pressure and/or flow of gas delivered to patient 10. However, it should be apparent that other techniques for controlling the pressure of the gas delivered to patient 10, such as varying the blower speed of gas flow generator 6, either alone or in combination with a pressure control valve, are contemplated by the present invention. Thus, valve 18 is optional depending on the technique used to control the pressure of the flow of breathing gas delivered to patient 10. If valve 18 is eliminated, the pressure generating system corresponds to gas flow generator 6 alone, and the pressure of gas in the patient circuit is controlled, for example, by controlling the motor speed of gas flow generator 6.

Pressure support system 2 further includes a flow sensor 22 that measures the flow of the breathing gas within delivery conduit 20 and delivery conduit 12. In the particular embodiment shown in FIG. 1, flow sensor 22 is interposed in line with delivery conduits 20 and 12, most preferably downstream of valve 18. Flow sensor 22 generates a flow signal, Q_{MEASURED}, that is provided to a controller 24 and is used by controller 24 to determine the flow of gas at patient 10 (Q_{PATIENT}).

Techniques for calculating Q_{PATIENT} based on Q_{MEASURED} are well known, and take into consideration the pressure drop of the patient circuit, known leaks from the system, i.e., the intentional exhausting of gas from the circuit as indicated by arrow E in FIG. 1, and unknown leaks from the system, such as leaks at the mask/patient interface. The present invention contemplates using any known or hereafter developed technique for calculating leak flow Q_{LEAK}, and using this determination in calculating Q_{PATIENT} based on Q_{MEASURED}. Examples of such techniques are taught by U.S. Pat. Nos. 5,148,802; 5,313,937; 5,433,193; 5,632,269; 5,803,065; 6,029,664; 6,539,940; 6,626,175; and 7,011,091, the contents of each of which are incorporated by reference into the present invention.

Of course, other techniques for measuring the respiratory flow of patient 10 are contemplated by the present invention, such as, without limitation, measuring the flow directly at patient 10 or at other locations along delivery conduit 12, measuring patient flow based on the operation of gas flow generator 6, and measuring patient flow using a flow sensor upstream of valve 18.

Controller 24 includes a processing portion which may be, for example, a microprocessor, a microcontroller or some other suitable processing device, and a memory portion that may be internal to the processing portion or operatively coupled to the processing portion and that provides a storage medium for data and software executable by the processing portion for controlling the operation of pressure support system 50, including automatically controlling humidity as described in greater detail herein.

An input/output device 26 is provided for setting various parameters used by airway pressure support system 2, as well as for displaying and outputting information and data to a user, such as a clinician or caregiver.

In the illustrated, non-limiting exemplary embodiment of the present invention, airway pressure support system 2 essentially functions as a CPAP pressure support system and pressure support device 4 provides functions of a CPAP base unit. Pressure support system 2, therefore, includes all of the capabilities necessary in such systems in order to provide appropriate CPAP pressure levels to patient 10. This includes receiving the necessary parameters, via input commands, signals, instructions or other information, for providing appropriate CPAP pressure, such as maximum and minimum CPAP pressure settings. It should be understood that this is meant to be exemplary only, and that other pressure support methodologies, including, but not limited to, BiPAP AutoSV, AVAPS, Auto CPAP, and BiPAP Auto, are within the scope of the present invention.

A scanning unit 30 is provided in pressure support device 4 and includes a sensor 32 for gathering data on one or more medical parameters associated with a non-sleep disordered breathing condition. Scanning unit 30 also includes a processing unit 31 to analyze the one or more medical parameters to determine a risk to the patient and an indicator 38 to provide an alert when the risk rises above a predetermined threshold level.

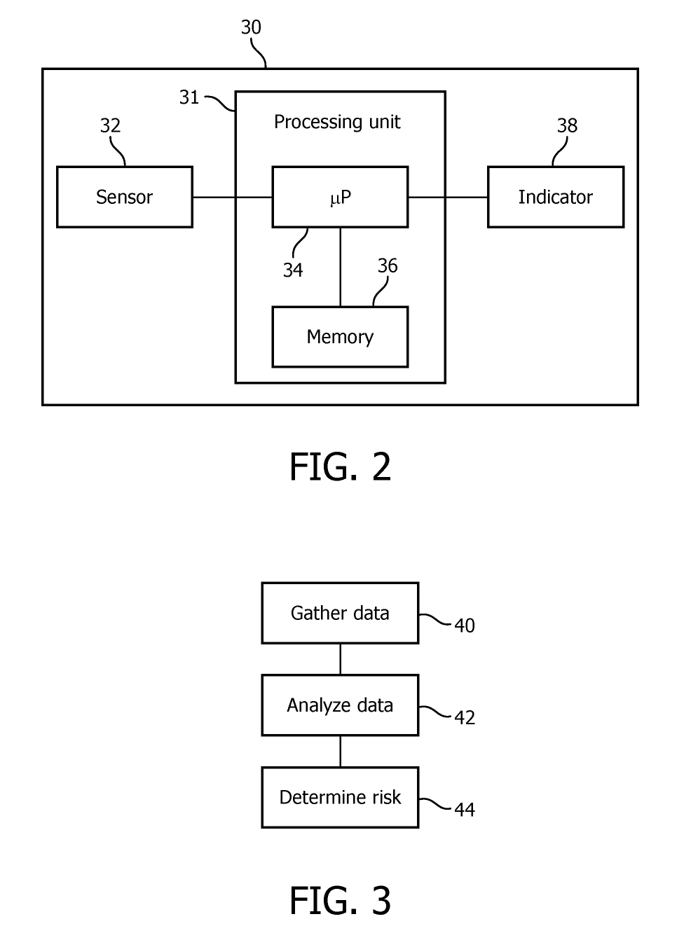

FIG. 2 is a schematic diagram of scanning unit 30 according to one particular, non-limiting exemplary embodiment. Scanning unit 30 includes sensor 32, processing unit 30, including a processor 34 and a memory 36, and indicator 38. Sensor 32 may be any type or can gather data on one or more medical parameters associated with a non-sleep disordered breathing condition from a patient. Examples of such conditions include, without limitation, a risk of pending cardiac arrest, Bradycardia, other cardiac arrhythmia, arterial stiffness, hypoxaemia, etc.

In one particular, non-limiting exemplary embodiment, sensor 32 is a spectroscopy based sensor. Spectroscopy is a known science and involves observing the resultant reflectance or absorption of energy when an object is exposed to electromagnetic radiation. The resultant reflectance or absorption of energy can indicate properties of the object. One application of spectroscopy in the healthcare field is pulse oximetry and in some exemplary embodiments, sensor 32 is structured to perform pulse oximetry. Absorption pulse oximetry involves passing two wavelengths of light through a body part to a photodetector. In reflectance pulse oximetry, the two wavelengths of light are not passed through the body part, but rather the photodetector is positioned to receive the reflected wavelengths off of the body part. Pulse oximetry is typically used to measure the oxygen saturation in a patient's blood.

Pulse oximetry is a fairly simple application of spectroscopy in healthcare, and spectroscopy based sensors employing pulse oximetry technology are fairly simple and inexpensive devices that generally employ two light sources such as, without limitation, light emitting diodes (LEDs) and a photodetector. However, it is contemplated that spectroscopy based sensors that utilize more advanced spectroscopy techniques may also be employed in exemplary embodiments of the disclosed concept.

For example and without limitation, spectroscopy based sensors that perform spatially offset Raman spectroscopy (SORS) or surface enhanced spatially offset Raman spectroscopy (SESORS) analysis may be employed as sensor 32 in exemplary embodiments of the disclosed concept. Spectroscopy based sensors employing SORS or SESORS may employed various components such as, without limitation, one or more lasers such as, without limitation, a variable frequency emitting laser with tunable repetition frequency such as a vertical cavity surface emitting laser (VECSEL) or multiple lasers with fixed frequency in conjunction with photodiodes or avalanche photodiodes. It is also contemplated that semiconductor saturable absorber mirrors (SESAMs), Raman lasers, and/or hyrbid silicon lasers may be employed in sensor 32 without departing from the scope of the disclosed concept. LEDs may also be employed in sensor 32 without departing from the scope of the disclosed concept. The use of SORS and SESORS allows for detection of parameters beyond oxygen saturation and blood. For example, SORS and SESORS may be used to detect bio-markers such as, without limitation, cardio bio-markers Cardiac Troponins T and I which are key markers associated with cardiac conditions. SORS and SESORS may also be used to detect markers such as C-reactive protein (CRP), which is a bio-marker identified in healthy patients, or Myoglobin, which is the first bio-marker after myocardial damage.

While pulse oximetry, SORS and SESORS spectroscopy based methods have been described in association with sensor 32, sensor 32 may be any type of spectroscopy based sensor suitable for use with any other spectroscopy based methods for determining medical parameters in a patient. Moreover, sensor 32 is not limited to spectroscopy based sensors, but rather may be any type of sensor suitable to use in determining a medical parameter associated with a non-sleep disordered breathing condition in a patient.

Sensor 32 is connected to processor 34, which in turn is connected to memory 36. Processor 34 may be, for example and without limitation, a microprocessor (μP), a microcontroller, or some other suitable processing device, that interfaces with memory 34 (which may be separate from or included as part of processor 36). Memory 36 can be any of one or more of a variety of types of internal and/or external storage media such as, without limitation, RAM, ROM, EPROM(s), EEPROM(s), FLASH, and the like that provide a storage register, i.e., a machine readable medium, for data storage such as in the fashion of an internal storage area of a computer, and can be volatile memory or nonvolatile memory. Memory 36 has stored therein a number of routines that are executable by processor 34. One or more of the routines store and/or analyze the output of sensor 32.

Referring to FIG. 3, a flowchart of a method of gathering and analyzing data in accordance with one particular, non-limiting exemplary embodiment is shown. The method of FIG. 3 may be employed, for example, with pressure support system 2 of FIG. 1 and/or scanning unit 30 of FIG. 2. At 40, data on one or more medical parameters is gathered from sensor 32 and stored in memory 36. The data may be gathered over time, such as on a daily basis. For example, a patient is likely to interact with pressure support system 2 on a regular basis in order to receive a pressure support therapy regimen. During or around the time of an interaction with pressure support system 2, such as during turning on or turning off pressure support system 2, sensor 32 can be used to gather data on one or more medical parameters.

At 42, the data is analyzed. The data is analyzed in order to identify a risk in the patient that is associated with the one or more medical parameters that are monitored. Any suitable technique may be used to analyze the data. In one particular, non-limiting exemplary embodiment, a baseline for the data is established over time and each new piece of data is analyzed to determine whether it substantially deviates from the baseline. A deviation in certain medical parameters can be indicative of a medical condition. For example and without limitation, statistical analysis may be performed on the gathered data to determine whether newly gathered data substantially deviates from the baseline value. In some example embodiments of the disclosed concept, the integration of statistical analysis with chemical analysis, which is occasionally referred to as chemometric techniques, may be employed to establish patterns with regression and prediction. In another particular, non-limiting exemplary embodiment, the data may be analyzed to determine whether certain patterns exist in the data. The existence of certain patterns in the data, such as the appearance of certain bio-markers, can be indicative of a medical condition. In some example embodiments, patterns of data from respiratory wave form analysis, pulse wave form analysis and potential bio-marker detection are integrated in analysis of the data. In some example embodiments, the analysis can determine a specific sequence or sequential characteristic pattern of bio-markers, subsequently appearing bio-markers and/or a duration of bio-markers. The integration with different types of data and regular monitoring for data can provide further improved detection of certain patterns that may deviate from a baseline and be indicative of a medical condition.

The use of chemometric techniques can discern the potential presence of noted bio-markers from other compounds that may be present in the body. Some other molecular compounds in the body may yield similar absorbance/reflectance for a given frequency. The complexity of SESORS provides an opportunity for label-free identification of such bio-markers and discerns them from other compounds. Moreover, data from sensor 32 combined with chemometric analysis can be employed in conjunction with pulse wave form analysis and respiratory wave form analysis for further enhancement in identifying a shift in medical parameters that may be indicative of a non-sleep disordered medical condition. The shift in medical parameters can confirm or validate the presence of a pattern or sequence in medical parameters which may aid in the calculation of a probability of risk of a non-sleep disordered medical condition in the patient.

Once the data is analyzed, a risk to the patient is determined at 44. The risk to the patient is based on the analyzed data and may be represented as a risk score. When the risk score exceeds a predetermined threshold, an indication such as, without limitation, an alarm or a message, may be provided to the patient via, for example and without limitation, indicator 38, to alert the patient of the risk.

Referring back to FIG. 2, indicator 38 is provided to alert the patient.

Indicator 38 may be any mechanism suitable to provide an alert to the patient such as, without limitation, a light source (e.g., without limitation, one or more light emitting diodes (LEDs), a liquid crystal display (LCD), etc.), a sound source or a combination of both light and sound sources. In some exemplary embodiments, indicator 38 may provide information to patient, such as text information or audible information. The information may be, for example and without limitation, a warning to consult a physician and/or information about which medical condition the patient may be at risk of.

In some exemplary embodiments, pressure support device 4 may monitor for emergency conditions such as, without limitation, when a patient stops breathing or when a patient's heart stops. The emergency condition may be monitored via scanning unit 30, for non-breathing related parameters, controller 24, for breathing related parameters, or a combination thereof. When an emergency condition is detected, pressure support device 4 may control indicator 38 to provide an indicator substantial enough that the patient or someone else in the vicinity of pressure support device 4, will notice the indication and take action. In some exemplary embodiments, the indication is a combination of visual and audible indications.

In some exemplary embodiments, indicator 38 may be omitted from scanning unit 30. Functions of indicator 38 may instead be incorporated into input/output device 26.

In one particular, non-limiting exemplary embodiment of the disclosed concept, scanning unit 30 is not as sophisticated or as expensive as advanced diagnostic equipment that may be found in a hospital. The capability of scanning unit 30 thus may not have the accuracy or detection capabilities of more advanced diagnostic equipment. However, by gathering data on one or medical parameters fairly regularly over time, the cumulative data set may be analyzed, thus allowing scanning unit 30 to more accurately identify a risk to the patient compared to if it was limited to only gathering data from the patient once.

In one particular, non-limiting exemplary embodiment, scanning unit 30 may not be able to precisely identify that the patient suffers from a medical condition associated with the monitored medical parameters. However, scanning unit 30 may be accurate enough to gauge the risk to the patient and indicate to the patient that the potential presence of the condition should be checked into further. The patient can then seek further evaluation by a physician or other healthcare professional.

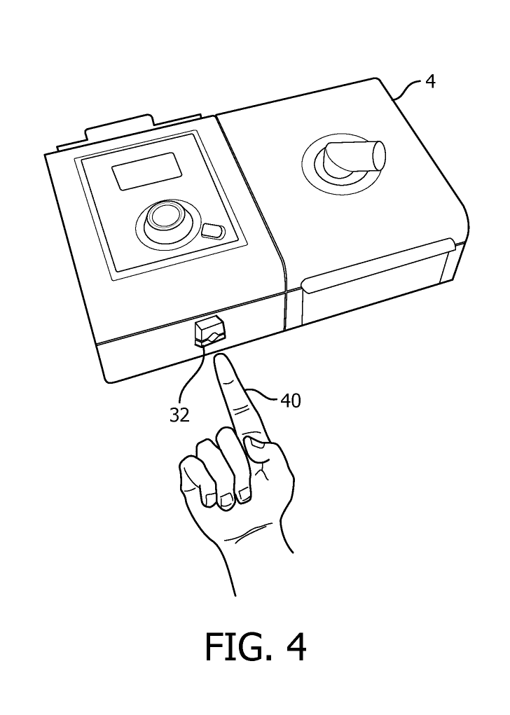

Referring to FIG. 4 an isometric view of pressure support device 4 in accordance with one particular, non-limiting exemplary embodiment is shown. Biometric sensor 32 may be disposed on or about the surface of pressure support device 4, as is shown in FIG. 4. In accordance with other exemplary embodiments of the disclosed concept, sensor 32 may be disposed outside of pressure support device 4 and be operatively connected to processing unit 31 via wires or other connection mechanisms. Sensor 32 may be employed to scan a portion of a patient, such as the patient's finger 40 to gather data on one or more medical parameters associated with a non-sleep disordered breathing medical condition.

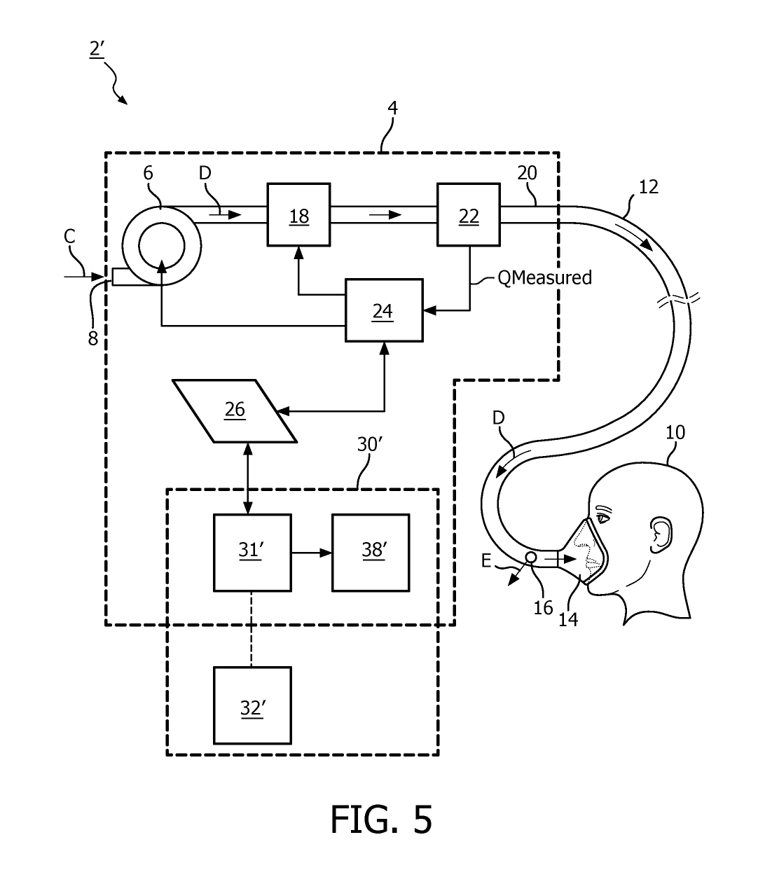

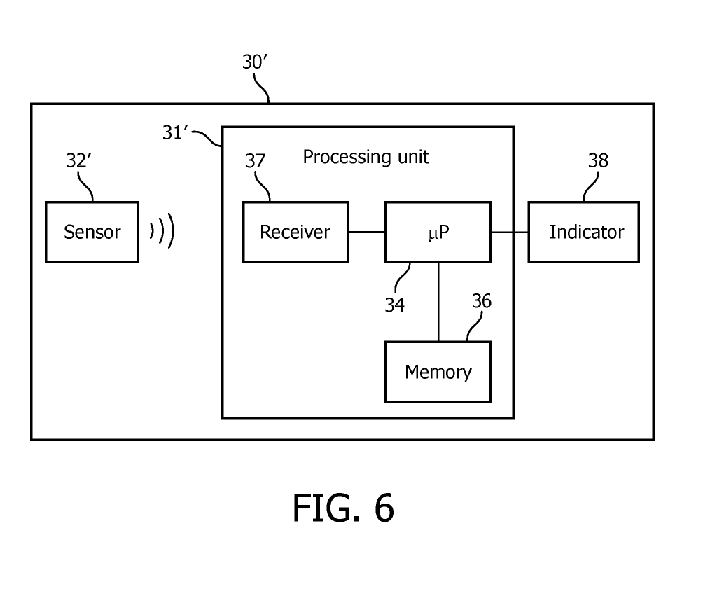

FIGS. 5 and 6 are, respectively, schematic diagrams of pressure support system 2′ and scanning unit 30′ in accordance with another exemplary embodiment. Pressure support system 2′ and scanning unit 30′ of FIGS. 5 and 6 are similar to pressure support system 2 and scanning unit of FIGS. 1 and 2. Description of similar elements is omitted for clarity and economy of disclosure. However, pressure support system 2′ and scanning unit 30′ of FIGS. 5 and 6 differs from pressure support system 2 and scanning unit 30 of FIGS. 1 and 2 in that sensor 32′ of scanning unit 30′ is structured to wirelessly communicate gathered data to processing unit 31′. To this end, processing unit 31′ includes a receiver 37 structured to receive the wirelessly transmitted data. The data may be wirelessly transmitted using any suitable wireless protocol such as, without limitation, Bluetooth.

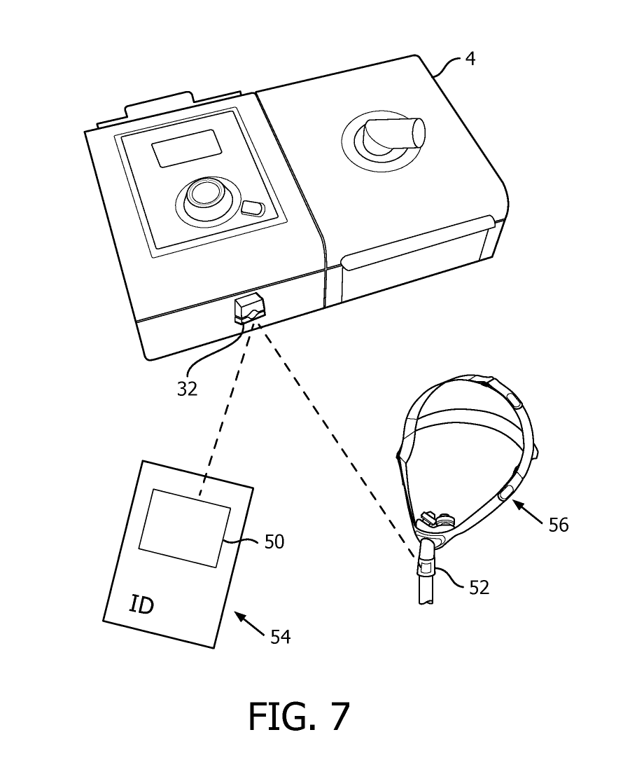

In addition to gathering data on one or more medical parameters associated with a non-sleep disordered breathing condition, sensor 32 may be employed to gather other information. Referring to FIG. 7, pressure support device 4 and sensor 32 in accordance with some exemplary embodiments may be employed to gather data from labels 50,52 on components such as, without limitation, a badge 54 or patient interface device 56.

As previously described, sensor 32 may be a spectroscopy based sensor.

Using spectroscopy, labels 50,52 can be obtained and a signature corresponding to compounds included in labels 50,52 may be obtained. The signatures corresponding to different compounds may be associated with pieces of information such as, without limitation, a part number, pressure support therapy settings, etc. For example, a database may be employed that associates particular signatures with particular pieces of information. Thus, by applying compounds to labels 50,52 that will produce intended signatures when labels 50,52 are scanned by sensor 32, information associated with the intended signatures can be retrieved when labels 50,52 are scanned.

In one particular, non-limiting exemplary embodiment, label 50 on badge 54 may include compounds that will produce a signature associated with pressure support therapy settings. A patient may carry badge 54 and scan label 50 with sensor 32. Pressure support device 4 may then retrieve the pressure therapy support settings associated with the signature that is produced when label 50 is scanned and automatically apply the pressure support therapy settings. In another particular, non-limiting exemplary embodiment, label 52 on patient interface device 56 may include compounds that will produce a signature associated with a part number of patient interface device 56. A patient may scan label 52 with sensor 32 and pressure support device 4 may then retrieve the part number of patient interface device 56 that is associated with the signature that is produced when label 52 is scanned. The part number may be useful, for example, to identify and/or re-supply equipment such as patient interface device 56.

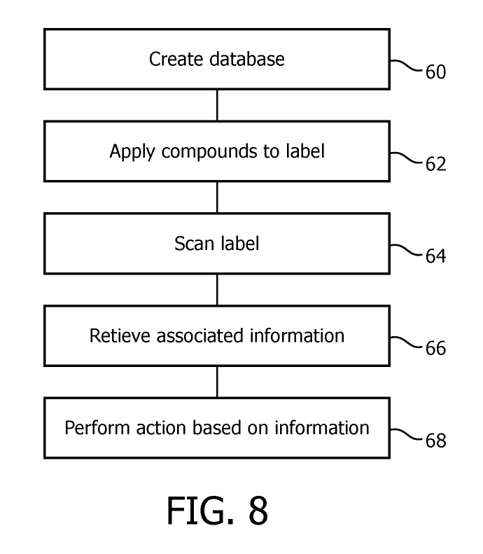

Referring to FIG. 8, a flowchart of a method in accordance with one particular, non-limiting exemplary embodiment is shown. The method of FIG. 8 may be employed, for example, in conjunction with pressure support device 4. At 60, a database is created. The database associates pieces of information with particular signatures. The signatures are signatures that may be obtained when sensor 32 is used to scan objects such as labels 50,52. At 62, compounds are applied to labels such as labels 50,52. The compounds are applied to the labels so as to produce an intended signature when the labels are scanned.

At 64, the labels, such as labels 50,52, are scanned by a sensor, such as sensor 32. When the labels are scanned, signatures associated with the compounds included in the labels are produced. At 66, information associated with the produced signatures is retrieved from the database. In some exemplary embodiments, database is located locally at pressure support device 4, such as in memory 36. However, database may also be located remotely from pressure support device 4, such as in a remote server, and pressure support device 4 may query database with information on the produced signature and receive in return the information associated with the produced signature.

At 66, action based on the obtained signature may optionally be performed. Such action may be varied. In some exemplary embodiments, the information is information on pressure support therapy settings and pressure support device 4 may adjust its pressure support therapy settings based on the information. For example and without limitation, processing unit 30 may apply the settings to pressure support device 4 upon receiving the information. By automatically adjusting the pressure support therapy settings based on the information, the patient does not need to manually adjust the settings. In some exemplary embodiments, the information may be part information, such as a part number or date information, and the information may be used for various reasons such as to identify the part, verify compatibility of the part, identify when a replacement for the part should be re-ordered, etc.

While some exemplary embodiments have been described in relation to how compounds on labels 50,52 can be used to encode information on labels 50,52 that may be retrieved by scanning labels 50,52 with sensor 32 on pressure support device 4, it is contemplated that the information encoded on labels 50,52 may also be retrieved by scanning labels 50,52 with other sensors similar to sensor 32. For example and without limitation, other sensors may be employed to scan labels 50,52 during manufacture and distribution of devices such as badge 54 and/or patient interface device 56 and the retrieved information may be used to, for example, identify the devices.

It is contemplated that aspects of the disclosed concept can be embodied as computer readable codes on a tangible computer readable recording medium. The computer readable recording medium is any data storage device that can store data which can be thereafter read by a computer system. Examples of the computer readable recording medium include read-only memory (ROM), random-access memory (RAM), CD-ROMs, magnetic tapes, floppy disks, and optical data storage devices.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A pressure support device (4) comprising:
a sensor disposed on an enclosure of the pressure support device and being structured to gather data on one or more medical parameters associated with a non-sleep disordered breathing medical condition;
a processing unit structured to analyze a risk to the patient of the non-sleep disordered breathing medical condition; and
an indicator structured to alert the patient when the risk exceeds a predetermined threshold level.

2. The pressure support device of claim 1, wherein the sensor is a spectroscopy based sensor.

3. The pressure support device of claim 2, wherein the sensor is structured to perform at least one of pulse oximetry, spatially offset Raman spectroscopy, and surface enhanced spatially offset Raman spectroscopy.

4. The pressure support device of claim 1, wherein the one or more medical parameters includes oxygen saturation in the patient's blood.

5. The pressure support device of claim 1, wherein the one or more medical parameters includes one or more bio-markers.

6. The pressure support device of claim 5, wherein the one or more bio-markers include at least one of cardiac troponin T, cardiac troponin I, C-reactive protein, and myoglobin.

7. The pressure support device of claim 1, wherein the non-sleep disordered breathing medical condition includes at least one of a risk of pending cardiac arrest, Bradycardia, cardiac arrhythmia, arterial stiffness, and hypoxaemia.

8. The pressure support device of claim 1, wherein the processing unit is structured to establish a baseline based on data gathered from the sensor over time and to analyze whether any new gathered by the sensor substantially deviates from the baseline.

9. The pressure support device of claim 1, wherein the processing unit is structured to analyze the data gathered from the sensor and to determine whether certain patterns exist in the data.

10. (canceled)

11. The pressure support device of claim 1, wherein the sensor is disposed outside an enclosure of the pressure support device and is structured to wirelessly communicate with the processing unit.

12. The pressure support device of claim 1, further comprising:
a gas flow generator structured to generate a pressurized flow of breathing gas for use in providing pressure support therapy to the patient.

13. The pressure support device of claim 1, wherein sensor is structured to scan a label and to produce a signature based on compounds included in the label, and wherein processing unit is structured to obtain information associated with the label from a database.

14. The pressure support device of claim 13, wherein the information includes pressure support therapy settings, and wherein processing unit is structured to apply the pressure support therapy settings to pressure support therapy device in response to retrieving the information.

15. The pressure support device of claim 1, wherein the indicator is structured to provide a visual and/or audible indication to alert the patient.
